(19) United States
(12) Patent Application Publication
(10) Pub. No.: US 2022/0136031 A1
(43) Pub. Date: May 5, 2022

(54) RECEPTACLE FOR DETECTION OF TARGETED PATHOGENS

(71) Applicant: MOBILEDETECT BIO INC., Sugar Land, TX (US)

(72) Inventors: Mark KISNER, Sugar Land, TX (US); Travis KISNER, Sugar Land, TX (US); Garrett PRESTON, Sugar Land, TX (US); Aaron SANDERS, Montgomery, TX (US); Mike GARRETSON, Sugar Land, TX (US); Courtney POWELL, Houston, TX (US)

(73) Assignee: MOBILEDETECT BIO INC., Sugar Land, TX (US)

(21) Appl. No.: 17/085,409

(22) Filed: Oct. 30, 2020

(51) Int. Cl. C12Q 1/04

(52) U.S. Cl. CPC C12Q 1/04

(57) ABSTRACT

A receptacle for detecting a targeted pathogen is provided. The receptacle includes a main body configured to accommodate reagent containing ampoules and a specimen collector, a reservoir provided at a distal end side of the main body, and a support extending through the main body in an extending direction of the receptacle. The support includes an inlet that permits passage of the specimen collector into the main body and an outlet that restrictively permits passage of the specimen collector into and out of the reservoir.

### BACKGROUND

### Field of the Disclosure

The present disclosure relates to the field of diagnostic detection of infectious pathogens, e.g., bacteria, fungi, parasites, and viruses, in humans, animals, and plants. More particularly, the present disclosure relates to a receptacle for the rapid collection and diagnostic detection of targeted infectious pathogenic agents that can be utilized in conjunction with a computing device, e.g., a portable computing device such as a smartphone, to rapidly analyze and determine the presence of and/or identity of the collected infectious pathogenic agent, and report the same to an appropriate regulatory body.

### Background information

The recent devastating spread of SARS-CoV-2, the virus that causes COVID-19 (i.e., the Coronavirus), has world nations, including the United States. scrambling for solutions to mitigate the continued spread of the disease. Similarly, unchecked animal and plant pathogens have been known to plague those populations as well (e.g., pathogens that affect livestock and crop populations). Testing for the presence of such infectious pathogens is a key aspect of any mitigation strategy. Testing permits identification of the pathogen, the possibility of immediate treatment, and contact tracing of populations that may have been exposed to or come into contact with an infected source. It also helps the appropriate public health officials, regulatory bodies, businesses, and individuals to coordinate and plan strategies within their communities and beyond to reduce the risk and stamp out the resulting diseases.

While efforts to control the spread of disease causing pathogens (e.g., the Coronavirus) are ongoing, effective and timely testing still faces many challenges. One of the obstacles to effective testing is the overwhelming demand for testing, which strains the supply chain, delays results, and exacerbates the problem to devastating effect. In addition, test kits and the procedures for detecting the presence of the disease also present barriers to obtaining rapid detection results. For example, a person concerned that they are exhibiting symptoms of the Coronavirus may go to a local hospital or health clinic for testing. Different testing facilities may have different types of tests to detect the same pathogen. Thus, different procedures must he followed to preserve the specimen until results can be interpreted. In such a scenario, the healthcare provider is trained to follow specific procedures to collect, preserve, store and transport the sample in a manner to prevent spoliation or contamination of the sample (along with potentially hundreds or thousands of other samples) until an approved laboratory or facility is able to complete the testing (assuming that the sample has survived each stop along its journey and has not been tampered with or compromised in some other manner) and determine whether or not the Coronavirus is present in the sample. Once the results are determined, that information must then be communicated back to the testing facility and/or the person who requested the test, which takes additional time and opens the door to more errors and delays.

Point-of-care test kits and home test kits are also available. However, even with the availability of such types of kits, receiving results can take anywhere from a few days to several weeks as the samples must not only be collected, they must also be preserved and shipped to approved facilities for testing. Thus, any perceived gains from existing point-of-care or home testing may be lost due to the time delay in obtaining the results with the unintended consequence of increased cases and the need for more testing.

Therefore, various groups, including those in the medical and public health field (e.g., physicians and veterinarians), the education field, law enforcement, military, entertainment industry (e.g., concerts, festivals, sporting events), food producers, and the health and fitness industry (es, gyms) have a need to detect the presence of targeted pathogens to inform and protect the health and safety of their groups, while also providing important information to the general public to help track and prevent the spread of the disease causing pathogens.

### SUMMARY OF THE INVENTION

Due to the high transmissibility of the disease causing pathogens, access to and location of testing is critical, as is preservation of the sample between collection and testing, and the timing to receive results. Thus, there is a need for a point-of-care (POC), e.g., at the point of sample collection, laboratory-grade diagnostic solution that meets the public health demand, is safe, easy to use, accurate, and minimizes concerns about contamination and tampering, while also enabling the much needed results to be obtained rapidly on-site and reported to the appropriate regulatory body anytime, anywhere (with smartphone automated detection and reporting technology).

Accordingly, according to aspects of the present disclosure, a receptacle for detecting a targeted pathogen is provided. The receptacle may include a main body configured to accommodate reagent containing ampoules and a specimen collector, a reservoir provided at a distal end side of the main body, and a support extending through the main body in an extending direction of the receptacle configured to receive reagents from ruptured reagent containing ampoules. The support may include an inlet that permits passage of the specimen collector into the main body and an outlet that restrictively permits passage of the specimen collector into and out of the reservoir.

According to aspects of the present disclosure, the main body may define a first chamber, a second chamber, and a third chamber. The second chamber and the third chamber may be disposed on opposite sides of the first chamber and may each be configured to accommodate one of the reagent containing ampoules. The support may be disposed in the first chamber, and the first chamber, the second chamber and the third chamber may be in fluid communication with the reservoir such that when the reagent containing ampoules accommodated in each of the second chamber and the third chamber are ruptured, the reagents are respectively released to flow into the reservoir.

According to other aspects of the present disclosure, the inlet of the support may include an entrance section and a restriction section. The restriction section may be disposed below the entrance section in a direction intersecting the extending direction of the receptacle, and the outlet may include a stopper section and a wringer section. The stopper section may be disposed in a direction facing a proximal end side of the main body and the wringer section may be disposed in a direction facing a proximal end side of the reservoir.

According to still other aspects of the present disclosure, the support may include a guide channel extending between the inlet and the outlet that is configured to guide the specimen collector through the main body and into and out of the reservoir.

According to additional aspects of the present disclosure, the support may be releasably attached to the main body so as to be insertable into and removable out of the receptacle.

According to aspects of the present disclosure, the support may include engagement members configured to engage a wall section disposed within the main body to secure the support within the main body, peripheral engagement sections projecting outwardly from each of the engagement members and configured to engage inner circumferential wail sections of a cap to secure the cap to the main body, and a projection projecting outwardly from the outlet and configured to contact the main body to align the outlet with an opening of the reservoir.

According to other aspects of the present disclosure, the main body may include a cover that is disposed along an upper side of the main body. The cover may include a deformation zone that permits resilient deformation of the cover toward an interior of the main body. The deformation zone may include materially thinned portions of the cover, and when the reagent containing ampoules are accommodated in the main body and an input force acting on the cover ruptures the reagent containing ampoules, the deformation zone may permit displacement of the cover towards the main body to trigger release of the reagents from the main body into the reservoir.

According to still other aspects of the present disclosure, the receptacle may include a removable cap provided to seal a proximal end side opening of the main body. The cap may include a stand section configured to stabilize placement of the receptacle in a vertically extending orientation and in a horizontally extending orientation.

According to additional aspects of the present disclosure, the support may include restriction plates provided on opposite sides of the guide channel to restrict the ruptured reagent containing ampoules from flowing with the released reagents from the main body into the reservoir.

According to further aspects of the present disclosure, a method of detecting a targeted pathogen is also provided. The method may include supplying the receptacle with a transfer medium and reagent containing ampoules accommodated in the main body, inserting the specimen collector containing a specimen into the main body through the inlet and the outlet of the support and into the reservoir to immerse the specimen in the transfer medium, removing the specimen collector from the reservoir through the outlet, applying an input force to deform the main body to rupture the reagent containing ampoules and release the reagents into the reservoir with the specimen immersed in the transfer medium, agitating the mixture of the released reagents, the specimen, and the transfer medium to incite a colorimetric reaction in the reservoir, and detecting the targeted pathogen based on a color of the colorimetric reaction.

According to aspects of the present disclosure, the method may also include, during the inserting of the specimen collector into the main body, passing the specimen collector through the entrance section of the inlet and the stopper section of the outlet, and during the removing of the specimen collector from the reservoir, pulling the specimen collector through the wringer section of the outlet so as to wring excess specimen laden transfer medium from the specimen collector back into the reservoir.

According to aspects of the present disclosure, the method may further include, during the applying of the input force to deform the main body, applying the input force to the cover such that the deformation zone permits displacement of the cover towards the main body to trigger the release of the reagents from the main body into the reservoir.

According to other aspects of the present disclosure, the method may include, prior to the inserting of the specimen collector containing the specimen into the receptacle, removing the cap from the main body to expose the proximal end side opening thereof to permit, the inserting of the specimen collector containing the specimen into the main body. The method may also include, after the removing of the specimen collector from the reservoir through the outlet, retaining the specimen collector between the inlet and the outlet of the support and securing the cap to the main body to seal the proximal end side opening and the specimen collector within the receptacle.

According to still other aspects of the present disclosure, the method may additionally include, after the specimen collector is pulled through the wringer section, locking the specimen collector into the restriction section of the inlet of the support, removing a breakable portion of the specimen collector extending from the restriction portion in the extending direction of the receptacle, and securing the cap to the main body to seal the receptacle and retain the specimen collector between the cap and the outlet of the support.

According to additional aspects of the present disclosure, the method may include, after the removing of the specimen collector from the reservoir through the outlet, securing the specimen collector to the support. sealing the receptacle with the specimen collector retained therein, inserting the reservoir into an incubator and heating the colorimetric reaction for a pre-determined amount of time, removing the reservoir from the incubator, detecting a color of the colorimetric reaction in the reservoir, and matching the color to a color guide to identify a presence or an absence of the targeted pathogen. In other aspects of the present disclosure and after removing the reservoir from the incubator, the method may further or alternatively include capturing, with an image processor of a computing device, an image of the colorimetric reaction in the reservoir to facilitate an automated colorimetric analysis performed by the computing device for detecting the targeted pathogen, and di splaying, on a display of the computing device, a result of the colorimetric analysis by the computing device indicating a presence of or an absence of the targeted pathogen.

According to still further aspects of the present disclosure, a support configured to be removably insertable into the receptacle for detecting the targeted pathogen is provided. The support may include an inlet that permits passage of a specimen collector into the receptacle and an outlet that restrictively permits passage of the specimen collector further into the receptacle. Engagement members may be disposed adjacent to the inlet and configured to engage wall sections disposed within the receptacle. Peripheral engagement sections may project outwardly from each of the engagement members and be configured to engage inner circumferential wall sections of the cap of the receptacle. Additionally, a guide channel may extend between and connect the inlet to the outlet to permit passage of the specimen collector into and out of the receptacle.

According to aspects of the present disclosure, the inlet may be a partition wall of the support and may include an entrance section and a restriction section. The entrance section may be a first opening of the partition wall and may be disposed at an upper side of the partition wall. The restriction section may be a second opening of the partition wall and may be disposed below the entrance section in a direction intersecting the extending direction of the guide channel. The first opening may be defined by downward sloping inclined upper wall sections of the partition, and the second opening may be in fluid communication with the first opening, be defined by downward extending side wall sections of the partition wall, and include a gate section and a retention recess.

According to other aspects of the present disclosure, the engagement members may each include a first side wall and a second side wall spaced from each other to define therebetween a slotted opening. The first side wall and the second side wall may be disposed in a parallel manner and project in the extending direction of the guide channel. for engaging within the slotted opening the wall sections disposed within the receptacle. The peripheral engagement sections may each include a back wail and a bottom wall adjacent to the second side wall of each of the engagement members. The back wall may be configured with an outer circumferential wall contoured for engaging inner circumferential wall sections of the cap of the receptacle. The bottom wall may include an upper surface and project in the extending direction of the guide channel. The guide channel may project from the inlet between the first side wall of each of the engagement members and include a track surface flanked on opposite sides by a first embankment wall and a second embankment wall.

According to still other aspects of the present disclosure, the outlet may include a first arcuate wall and a second arcuate wall configured in an open ring shape having a through hole to restrictively permit passage of the specimen collector therethrough, and the first arcuate wall and the second arcuate wall may define a stopper section on a proximal end side of the outlet and a wringer section on a distal end side thereof.

According to additional aspects of the present disclosure, the first embankment wall and the second embankment wall may each include a cut part and the outlet is integrally formed at the cut part. The outlet may include a first arcuate wall and a second arcuate wall configured in an open ring shape axially aligned with the guide track to restrictively permit passage of the specimen collector therethrough, and the first curved wall and the second curved wall may define a stopper section on a proximal end side of the outlet and a wringer section on a distal end side thereof.

### BRIEF DESCRIPTION OF THE DRAWINGS

FIG. 1 is an exemplary perspective view of a receptacle, according to an aspect of the present disclosure.

FIG. 2 is an exemplary inverted perspective view of the receptacle, according to an aspect of the present disclosure.

FIG. 3A and FIG. 3B are exemplary upright perspective views of two receptacles each showing a variation of a detachable cap, according to aspects of the present disclosure.

FIG. 4 is an exemplary top side perspective view of an inside of the receptacle with a cover removed and a support disposed therein, according to an aspect of the present disclosure.

FIG. 5 is an exemplary cross-sectional view of the receptacle with the support disposed therein when viewed from a proximal end side of the receptacle towards a distal end side thereof, according to an aspect of the present disclosure.

FIG. 6A is an exemplary top plan view of a cover of the receptacle, according to an aspect of the present disclosure.

FIG. 6B is an exemplary upright perspective view of the cover of the receptacle illustrating displacement of the cover due to a deformation force acting on the same, according to an aspect of the present disclosure.

FIG. 7A is an exemplary perspective view of the support, according to an aspect of the present disclosure.

FIG. 7B is an exemplary top side perspective view of the support, according to an aspect of the present disclosure.

FIG. 8 is an exemplary bottom side perspective view of an inside of the receptacle with a base housing removed and the support disposed thereon, according to an aspect of the present disclosure.

FIG. 9 is an exemplary inverted cross-sectional view of the receptacle with the support disposed therein when viewed from an interior of the receptacle towards the proximal end side thereof, according to an aspect of the present disclosure.

FIG. 10 is a partial perspective view of a distal end side of the receptacle and an outlet of the support, according to an aspect of the present disclosure.

FIG. 11A is an exemplary perspective view of another support, according to an aspect of the present disclosure.

FIG. 11B is an exemplary cross-sectional view of the support shown in FIG. 11A, according to an aspect of the present disclosure.

FIG. 12A shows an exemplary process for initiating a targeted pathogen detection operation with the receptacle, according to an aspect of the present disclosure.

FIG. 12B shows an exemplary process for carrying out the targeted pathogen detection operation with the receptacle, according to an aspect of the present disclosure.

FIG. 13 illustrates an exemplary intermediate state of the receptacle during the targeted pathogen detection operation showing reaction fluid in a reservoir of the receptacle just prior to an incubation period, according to an aspect of the present disclosure.

FIG. 14A illustrates an exemplary incubation state of a plurality of receptacles during the incubation period, according to an aspect of the present disclosure.

FIG. 14B illustrates an exemplary incubation state of the plurality of receptacles during the incubation period, according to another aspect of the present disclosure.

FIG. 15 illustrates an exemplary result capturing state of the receptacle in which a mobile computing device captures an image of the colorimetric reaction to analyze and detect the presence of the targeted pathogen, according to an aspect of the present disclosure.

### DETAILED DESCRIPTION

In view of the foregoing, the present disclosure, through one or more of its various aspects, embodiments and/or specific features or sub-components, is thus intended to bring out one or more of the advantages as specifically noted below.

Methods described herein are illustrative examples, and as such are not intended to require or imply that any particular process of any embodiment be performed in the order presented. Words such as “thereafter,” “then,” “next,” etc. are not intended to limit the order of the processes, and these words are instead used to guide the reader through the description of the methods. Further, any reference to claim elements in the singular, for example, using the articles “a,” “an” or “the”, is not to be construed as limiting the element to the singular.

As will be discussed below as non-limiting embodiments of the present application, a receptacle, a support, and a related method for the detection of targeted pathogens using the receptacle and support is provided. The receptacle and support in conjunction with the method enables manual or automated colorimetric detection of the targeted pathogens, as well as automated post-analysis reporting and evidence collection functionality. Such a system enhances the ability of its users to rapidly and accurately detect at the point-of-care targeted pathogens of interest while avoiding complex and lengthy lab procedures, mishandling/contamination of samples between collection and results interpretation, and long wait, times for testing, results, reporting and other administrative documentation. The reporting functionality allows a user to transmit/export results, reports and other necessary documentation over a communications network, as desired.

In embodiments, an application discussed herein uses a smartphone camera to detect the color of a chemical reaction. While a smartphone camera is exemplary, it is also contemplated that any other imaging device connected to, e.g., a laptop, a watch, a pair of (glasses, a networked security camera (and/or that is wired or wirelessly paired, e.g., to the smartphone) may be employed to capture the colorimetric reaction for analysis by the application.

While the testing application is configured to detect various disease causing pathogens in humans, animals, and plants, it is contemplated that the testing application may also be configured to analyze any number of chemical colorimetric reactions for detecting, e.g., explosives, including both military and commercial grade, drugs, other bio-hazards, homemade explosives HMEs) and toxins.

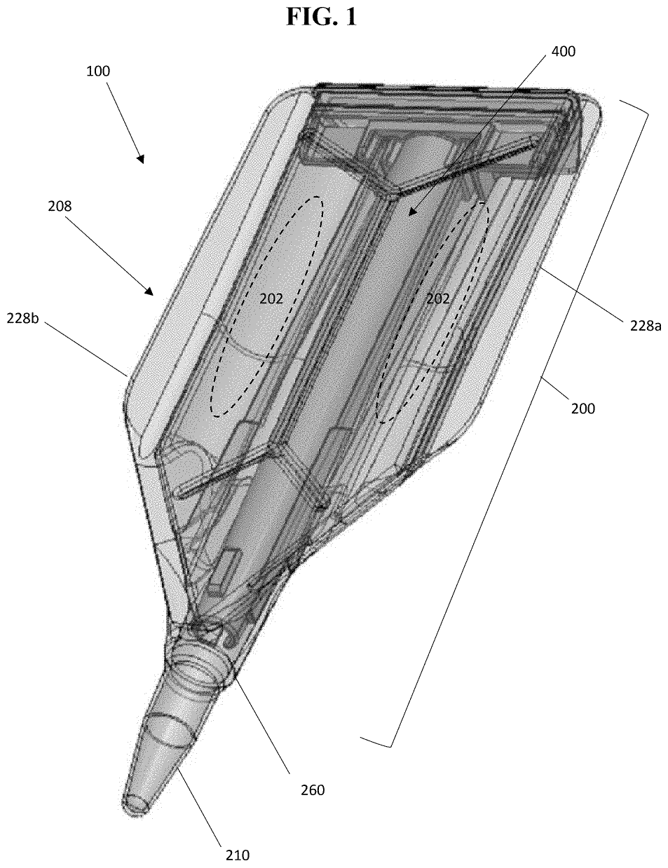

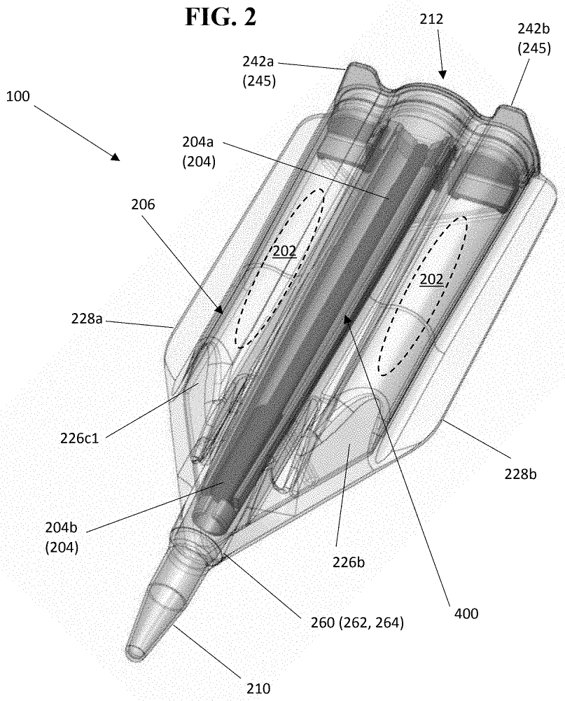

FIGS. 1 and 2 illustrate exemplary perspective views of a receptacle 100 designed to facilitate detection of a targeted pathogen. The entire receptacle 100 is packed into a small form factor for portability and handling. The receptacle 100, also referred to as a pouch or a container, includes a main body 200 configured to accommodate reagent containing ampoules 202 and a specimen collector 204. The specimen collector 204 includes a handle 204a and an applicator swab 204b. The specimen collector 204 may be, for example, any one of a nasopharyngeal, nasal, or oropharyngeal swab. The receptacle 100 may he injection molded using any suitable polymer plastic having sufficient strength, puncture resistance, and elasticity so as to prevent puncture of the same even when the receptacle 100 is manipulated by the user and the ampoules 202 are ruptured. In addition, the receptacle 100 is configured to enhance shock absorption so as to prevent inadvertent ampoule 202 breakage during storage, transport or handling prior to use in a targeted pathogen detection operation.

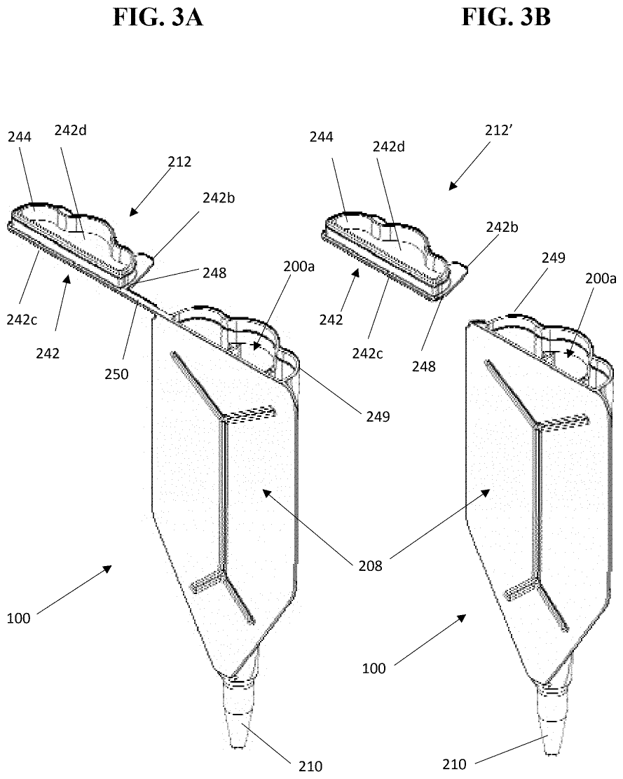

The main body 200 includes a base housing 206 and a cover 208 disposed at a top side of the base housing 206. A reservoir 210 is disposed at a distal end side of the main body 200 and a removable cap 212 is provided at a proximal end side of the main body 200. As shown in FIG. 3A, the removable cap 212 may be connected to the main body 200 in a manner that prevents separation of the cap 212 from the main body 200 even when removed from the opening of the main body 200. Alternatively and as shown in FIG. 3B, the removable cap 212′ may be completely separable from the main body 200 when removed from the opening of the same.

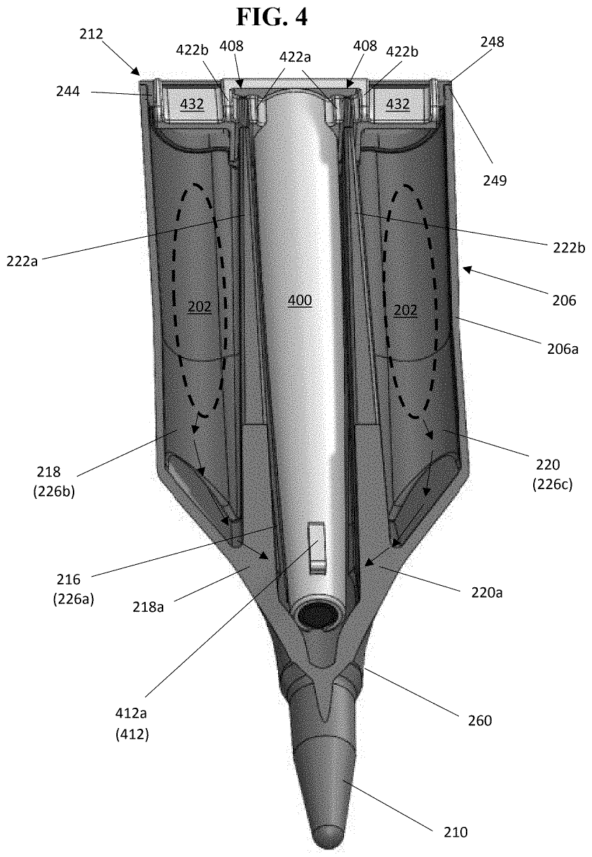

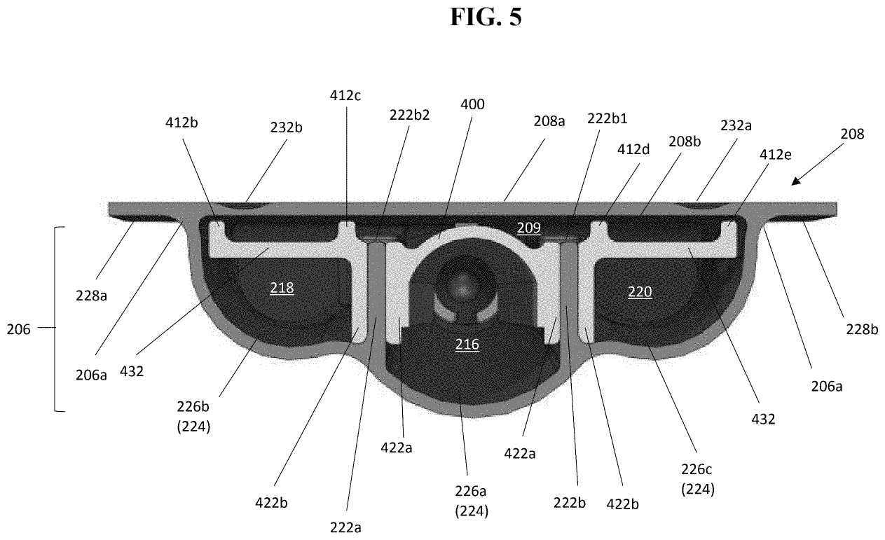

As shown in FIGS. 4 and 5, the base housing 206 is recessed to define an interior space within the main body 200. The base housing 206 defines a first chamber 216 extending through a central region of the main body 200, and a second chamber 218 and a third chamber 220 disposed on opposite sides of the first chamber 216. The first, second and third chambers 216 218, and 220 extend along an extending direction of the receptacle 100 between the removable cap 212 disposed at the proximal end side of the main body 200 and the reservoir 210 disposed at the distal end side of the receptacle 100. A volume of the first chamber 216 is larger than a volume of each of the second and third chambers 218, 220, respectively, and the chamber 216 is disposed between the second and third chambers 216, 218 in a stepped manner such that the first chamber 216 extends further from the cover 208 than the second and third chambers 218, 220 in a direction orthogonal to the extending direction of the receptacle 100.

As shown in FIG. 5, two ribs 222a and 222b project upward from a bottom inner surface 224 of the base housing 206 in a projecting direction towards the cover 208. As shown in FIG. 4, the ribs 222a and 222b also extend from the proximal end side of the main body 200 to the distal end side thereof alongside fin the extending direction of) the first, second, and third Chambers 216, 218, and 220. In particular, rib 222a extends in the extending direction of the receptacle 100 between the first chamber 216 and the second chamber 218 to partition the first chamber 216 from the second chamber 218 in a direction intersecting the extending direction of the receptacle 100. Rib 222b extends in the extending direction of the receptacle 100 between the first chamber 216 and the third chamber 220 to partition the first chamber 216 from the third chamber 220 in the direction intersecting the extending direction of the receptacle 100.

The second chamber 218 and the third chamber 220 are each configured to receive the reagent containing ampoules 202. When the reagent containing ampoules 202 are disposed within the main body 200, the ribs 222a and 222b prevent the reagent containing ampoules 202 in each of the second chamber 218 and the third chamber 220 from being displaced into the first chamber 216. While the ribs 222a and 222b extend upward in the projecting direction from the bottom inner surface 224 of the main body 200, the ribs 222a and 222b are spaced from the cover 208 to permit deformation of the cover 208 back towards the main body 200 to allow rupture of the ampoules 202 diming the targeted pathogen detection operation. The spacing between the cover 208 and the ribs 222a and 222b also permits the receptacle 100 to be injection molded, as well as to facilitate the aggregation and mixing of the reagents with the transfer medium prior to incubation.

Due to their height and extending length along the receptacle 100, the ribs 222a and 222b not only retain the ampoules 202 in their respective chambers (during manufacture/assembly of the receptacle 100, during transport and storage, and during operational handling prior to rupturing), but also restrict shards of glass or plastic from ruptured ampoules 202 from entering into other portions of the receptacle 100, e.g., the first chamber 216 and the reservoir 210. The ribs 222a and 222b also control fluid flow (directional flow and flow rate) of the reagents released from ruptured ampoules 202 toward the reservoir 210, provide structural rigidity to the base housing 206, and limit the amount of displacement of the cover 208 toward the bottom inner surface 224 of the base housing 206 when a force acts to deform the same during the targeted pathogen detection operation.

As shown in FIG. 4, the first chamber 216 extends further through the main body 200 than the ribs 222a and 222b and the second and third chambers 218 and 220. The first chamber 216, the second chamber 218, and the third chamber 220 are each respectively defined by an arcuate channel 226a, 226b, and 226c formed in the base housing 206. Collectively, the arcuate channels 226a, 226b, and 226c define the bottom inner surface 224 of the main body 200. The arcuate channels 226a, 226b, and 226c taper as they extend from the proximal end side of the main body 200 to the distal end side thereof. The arcuate channel 226a narrows along a central axis of the same from the proximal end side of the main body 200 to the distal end side thereof. The arcuate channels 226b and 226c each include a slope section 226b1 and 226c1, respectively. Each slope section 226b1 and 226c1 respectively includes an inclined surface that inclines from its respective arcuate channel toward the cover 208 while also tapering towards the central region of the main body 200 to respectively form, with a distal side end of each rib 222a and 222h, flow channels 218a and 220a for the reagents to flow from the second and third chambers 218 and 220 to a distal end side of the first chamber 216. The flow channels 218a and 220a control the fluid flow of the reagents from the second and third chambers 218, 220 into the distal end side of the first chamber 216 for dispensing into the reservoir 210. The flow channels 218a, 220a thus permit fluid communication between the distal end side of the first chamber 216 and distal end sides of the second and third chambers 218, 220.

The first chamber 216 extends toward the reservoir 210 such that the first chamber 216 is in concentric alignment with the reservoir 210 in the extending direction of the receptacle 100. As will be discussed in detail below, the first chamber 216 may be configured to receive a support 400 that is insertable through the first chamber 216 from the proximal end side of the main body 200 to the distal end side thereof. It is contemplated, however, that the features of the support 400 may be integrally provided in the receptacle 100.

As shown ire FIG. 5, the cover 208 is disposed over an upper end side of the base housing 206 opposite the bottom inner surface 224 of the main body 200. As shown in FIG. 1, the cover 208 spans the lengths of the first chamber 216, the second chamber 218, and the third chamber 220 from the proximal end side of the main body 200 to the distal end side of the main body 200. The cover 208 also includes flanged portions 228a and 228b that project outward from upper side surfaces 206a of the base housing 206 in a direction orthogonal to the extending direction of the receptacle 100. The flanged portions 228a and 228b enhance grip-ability of the receptacle 100 with one hand during handling or during the targeted pathogen detection operation. The flanged portions 228a and 228b also define areas in which indicia. (including, e.g., information containing labels) can be placed and serve as alignment indicators during manufacturing for purposes of, e.g., confirming label placement and locating/conveying the receptacle 100 along the manufacturing line.

The upper side surfaces 206a define outer upper circumferential outer portions of the second chamber 21$ and the third chamber 220. The cover 208 extends over the outer upper circumferential outer portions of the second chamber 218 and the third chamber 220 from the proximal end side of the main body 200 to the distal end side of the main body 200. The flanged portions 228a and 228b project outwardly from the outer upper circumferential outer portions 206a. The cover also begins to taper toward the central region of the main body 200 at an area adjacent the slope sections 226b1 and 226c1 such that opposite outer sides of the cover 200 converge (from the flanged portions 228a and 228b) towards an upper distal end side of the first chamber 216. The tapered configuration of the cover 208 minimizes material use, as well as minimizes the possibility of interference between the cover 208 and an incubator into Which the reservoir 210 is inserted during the targeted pathogen detection operation. The tapered configuration of the cover 208 also reduces space consumption when multiple receptacles 100 are laterally stacked (at an offset 180° orientation) in storage or in preparation of use.

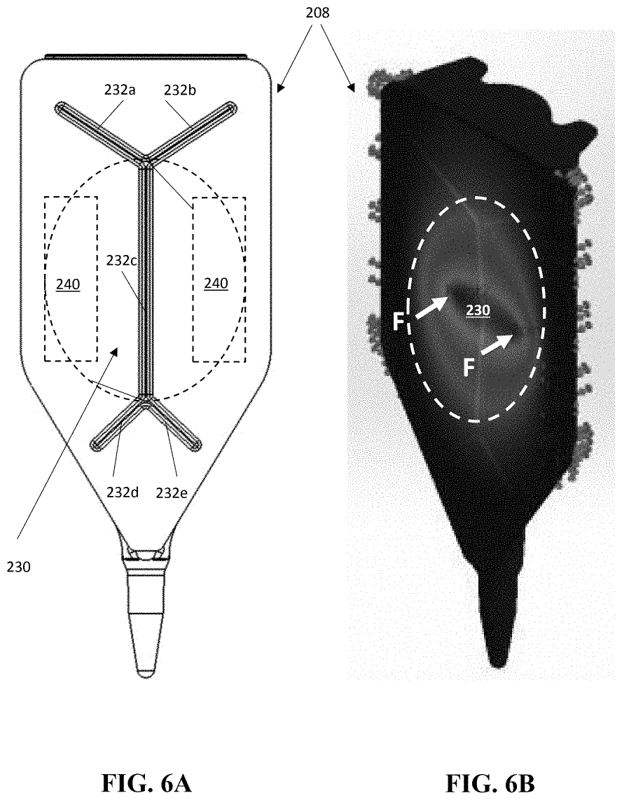

As shown in FIG. 6A, the cover 208 includes a deformation zone 230 that permits controlled resilient deformation of the cover 208 toward an interior of the main body 200. The deformation zone 230 includes a plurality of materially thinned portions 232a, 232b, 232c, 232d, and 232e of the cover 208 that appear as recessed grooves in an upper surface 208a of the cover 208. Materially thinned portion 232c is disposed in the central region of the main body 200 at a position on the cover 208 above and axially aligned along a central axis of the first chamber 216. The materially thinned portion 232c extends along the cover 208 in the same extending direction as the first chamber 216 and includes a first joint section 232c1 disposed closer to the cap 212 than the reservoir 210 and a second joint section 232c2 disposed closer to the reservoir 210 than to the cap 212. Materially thinned portions 232a and 232b extend outwardly in opposite directions from the first joint section 232c1 towards the proximal end side of the main body 200. In embodiments, the materially thinned portions 232a and 232b each extend outwardly at a 45° angle from the first joint section 232c1. The materially thinned portions 232a and 232b each extend from an area of the cover 208 directly above the first chamber 216 to an area of the cover 208 directly above the second and third chambers 218 and 220, respectively.

Materially thinned portions 232d and 232e extend outwardly in opposite directions from the second joint section 232c2 towards the distal end side of the main body 200. In embodiments, the materially thinned portions 232d and 232e each extend outwardly at a 45° angle from the second joint section 232c2. The materially thinned portions 232d and 232e each extend from an area of the cover 208 above the first chamber 216 to an area of the cover 208 directly above the second and third chambers 218 and 220, respectively.

The mater ally thinned portions 232a, 232b, 232c, 232d, and 232e are designed to extend along areas of the cover 208 outside of ampoule holding regions (or input force receiving zones) 240 of the second and third chambers 218 and 220. This arrangement enables an input force F to act on an area of the cover 208 outside of, or overlapping, the deformation zone 230, which in turn increases the number of possible locations in which the input force F can be applied. It is noted that the input force receiving zone 240 is an area of the cover 208 that includes materially thick portions. The materially thick portions are thicker than the materially thinned portions 232a, 232b, 232c, 232d, and 232e and, as discussed previously, are sufficiently thick to prevent shards of ruptured glass or plastic to penetrate the cover 208. Such a design protects users of the receptacle 100 from cuts, puncture wounds, and potential exposure to the targeted pathogen being tested even if excess input force F is exerted on the cover 208. The arrangement of the materially thinned portions 232a, 232b, 232c, 232d, and 232e also identities to the user the locations of the input force receiving zones 240 for placement of fingers (e.g., thumbs) on the cover 208 to rupture the ampoules 202.

Additionally, the positioning of the ribs 222a and 222b, i.e., extending upward towards the cover 208 from the bottom inner surface 224 of the first chamber 216, limits the amount of displacement of the cover 208 when the input force F acts to deform the same during the targeted pathogen detection operation. If the input force F exceeds a threshold amount, a bottom surface 208b of the cover 208 (facing the interior of the main body 200) contacts an upper edge surface 222a1 and 222b1 of the ribs 222a and 222b to stop further deformation and structural failure of the receptacle 100 (e.g., cracking and/or breakage of any part of the cover 208 main body 208, reservoir 210, cap 212, and/or support 400).

As shown in FIG. 6B, when the reagent containing ampoules 202 are accommodated in the main body 200 and an input force F acts on the deformation zone 230, the deformation zone 230 permits displacement of the cover 208 towards the main body 200 to trigger release of the reagents from the ampoules 202 through the second and third chambers 218 and 220, respectively, and into the reservoir 210. That is, during deformation the bottom surface 208b of the cover 208 is forced into the reagent containing ampoules 202 such that the ampoules 202 are ruptured and the reagents flow toward the reservoir 210.

As also shown in FIG. 6B, when the input force F is applied to the input force receiving zone 240, the materially thinned portions 232a, 232b, 232c. 232d, and 232e permit downward. displacement of the cover 208 toward the main body 200, i.e., permits the deformation zone 230 of the cover 208 to collapse or bend downwardly and inwardly at the weakened portions toward the interior of the main body 200. Such a construction permits sufficient rigidity to the receptacle 100 for purposes of transport, storage and handling, while readily enabling reliable deformation of the cover 208 to ensure consistent rupturing of the reagent containing ampoules 202 in the second and third chambers 218 and 220 and release of the reagents towards the reservoir 210 for carrying out the targeted pathogen detection operation.

As shown in FIGS. 3A, 3B and 4, the cap 212 is removably provided to seal a proximal end side opening 200a of the main body 200. The cap 212 includes a seal plate 242 and a seal protrusion 244. The seal plate 242 includes leg sections 242a and 242b that project outwardly from end sides of a top surface 242c of the seal plate 242. The leg sections 242a and 242b are tapered projections that extend outwardly from the seal plate 242 in the same direction. The leg sections 242a and 242b project from the seal plate 242 a length that is at least equal to or farther than other sections of the seal plate 242 projecting in the same direction.

The seal plate 242 and the leg sections 242a and 242b define a stand section 245. The stand section 245 stabilizes placement of the receptacle 100 in both of a vertically extending orientation or a horizontally extending orientation. The top surface 242c of the seal plate includes sufficient surface area (including top surface portions of the leg sections 242a and 242b) to support the receptacle 100 in an upright position in the vertically extending direction.

The leg sections 242a and 242b also support the receptacles 100 in a variety of horizontal positions (in the horizontally extending orientation) in a secure and balanced manner even when the receptacles 100 are placed on an uneven or graded sloped surface. Such a construction enhances the versatility of the receptacle 100 in the field or at the point of care for storage, organization, and convenience irrespective of the environment in which the receptacle is used.

It is also contemplated that the stand section 245 may he configured to support or facilitate stacking and/or nesting of multiple receptacles 100 positioned in the horizontally extending orientation, e.g., when any two adjacent horizontally positioned receptacles 100 are vertically stacked at an offset 180° orientation.

The seal protrusion 244 projects from a bottom surface 242d of the seal plate 242 and in a direction generally orthogonal to the leg sections 242a and 242b (i.e., in the extending direction of the receptacle). The seal protrusion 244 is contoured to compliment the shape of the opening of the proximal end side of the main body 200 and configured to engage an inner peripheral surface of the main body 200 within the opening. The seal protrusion 244 and the inner peripheral surface of the main body 200 engage each other in a releasable friction-fit or releasable snap-fit seal manner to prevent liquid, ampoules, glass/plastic shards, and specimen collectors from leaking or being removed once the cap 212 is secured to the main body 200.

The seal protrusion 244 is also disposed at a position inward from outer peripheral portions of the bottom surface 242d of the seal plate 242. The outer peripheral portions define cap seal edges 248 that are configured to abut or contact corresponding receptacle seal edges 249 of the main body 200. The receptacle seal edges 249 define the opening into the main body 200. The leg portions 242a and 242b extend beyond the cap seal edges 248 and when the cap 212 is attached to the main body 200, the leg portions 242a and 242b serve as grips or handle tabs to conveniently and assuredly pull the cap 212 from the main body 200 to open the receptacle 100 for access into the interior of the main body 200.

A tether strap 250 flexibly connects the cap 212 to the cover 208 of the main body 200. In embodiments, the tether strap 250 is an integrally provided living hinge 250 that extends from the cover 208 to connect the cap 212 to the same. It is contemplated that the tether strap 250 could be connected to other portions of the receptacle 100 without departing from the spirit and the scope of the application, e.g., placement on the base housing 206. The tether strap 250 prevents the cap 212 from being separated from the receptacle 100, misplaced or inadvertently discarded. This enhances efficiency during testing and minimizes opportunities to inadvertently compromise tests, which would otherwise lead to inaccurate tests and/or testing delays. In embodiments and as shown in FIG. 313, the cap 212 may be entirely separable. In addition, the tether strap 250 enables the receptacle 100 and the cap 212 to be formed as a single injection molded part for manufacturing simplicity.

The reservoir 210, disposed at the distal end side of the main body 200, integrally extends from the first chamber 216 at a connection section 260 which is at a distance spaced from a distal end of the cover 208 in the extending direction of the receptacle 100. The reservoir 210 is a tubular container having a tapered or conical distal end although it is contemplated that the reservoir may be configured in a variety of shapes without departing from the spirit and scope of the application. It is also contemplated that a volume of the reservoir 210 is larger than a combined volume of the reagents contained in the ampoules 202 and the transfer medium contained within the receptacle 100 so as to be of sufficient size to accommodate the reagents and transfer medium during the targeted pathogen detection operation. It is further contemplated that the volume of the reservoir 210 is also of sufficient size to retain the reagent/transfer medium mixture below the connection section 260 between a proximal end side of the reservoir 210 and the distal end side of the first chamber 216.

A collection opening 262 of the reservoir 210, provided at the proximal end thereof, is in fluid communication with a discharge opening 264 of the first chamber 216 at the connection section 260. In embodiments, the transfer medium is pre-filled into the receptacle 100. For example, it is contemplated that the transfer medium is injected into the receptacle 100 during assembly of a detection kit and sealed when the cap 212 is attached to the opening of the main body 200. Thus during storage and transport, the transfer medium is able to flow freely throughout the interior of receptacle 100 prior to the targeted pathogen detection operation. In other embodiments it is contemplated that the transfer medium is disposed in the reservoir 210 and retained therein via a frangible seal (hermetic or otherwise) that can be ruptured due to a puncture force and/or a pressure force by the specimen collector 204. In further embodiments, the transfer medium is pipetted into the receptacle 100 just prior to an initiation of the targeted pathogen detection operation. The transfer medium may be any suitable liquid or medium, such as any known viral transport medium, saline, or semi-fluid that can preserve, augment or enable testing of the collected pathogenic samples from humans, animals, and plants.

The reservoir 210 is designed to be readily mounted in an incubator, e.g., a heater or thermal cycler and may be a transparent, scratch/puncture resistant, and heat resistant polymer plastic. Thus in embodiments, it is contemplated that the reservoir 210 is formed as a cuvette insertable into a well provided on a heater block of the heater or thermal cycler.

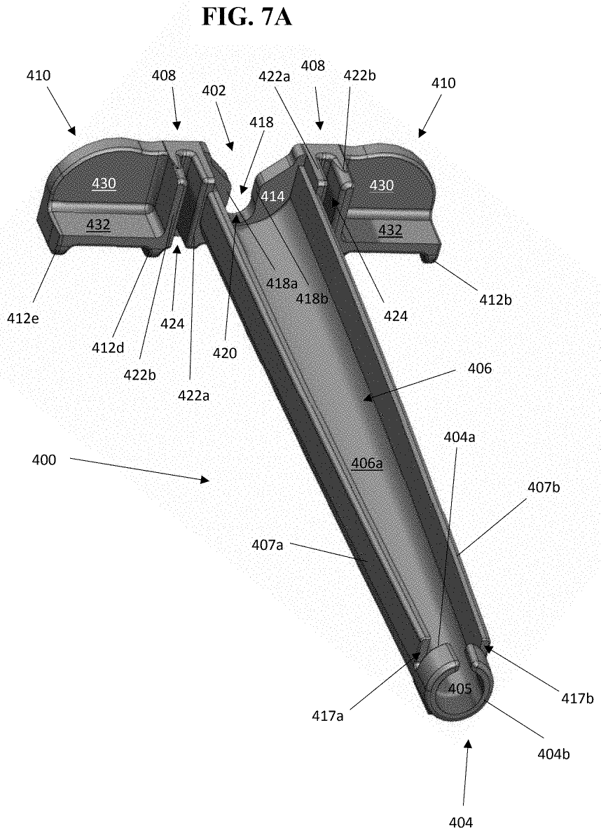

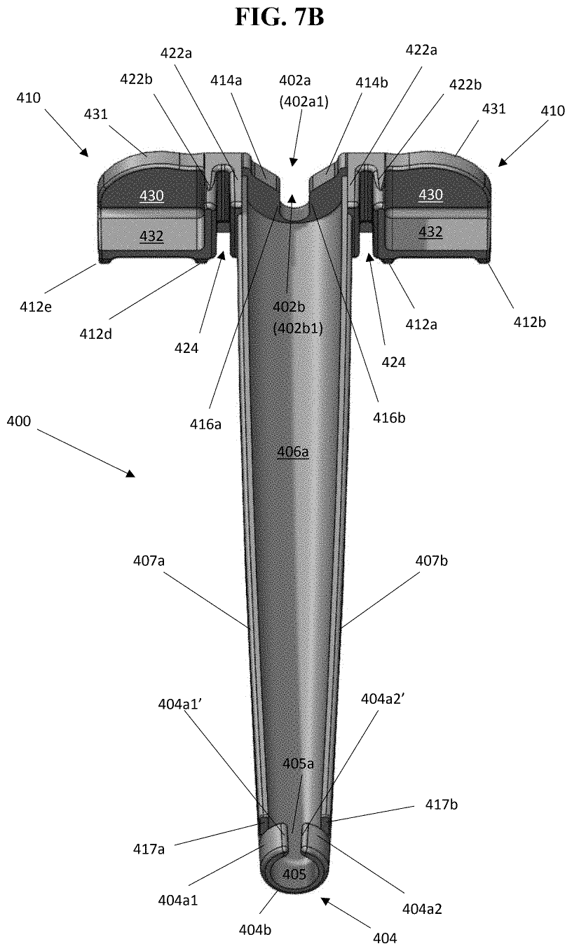

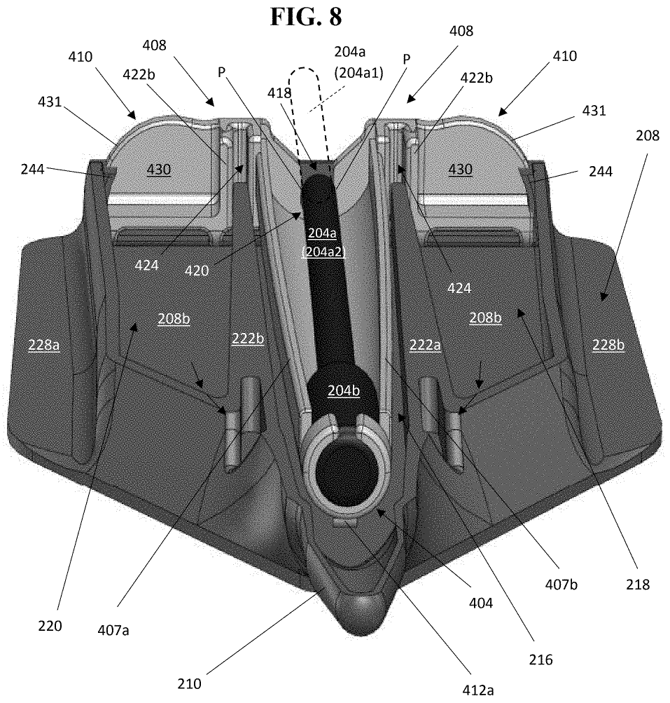

As shown in FIGS. 7A, 7B, and 8 the receptacle 100 is configured to receive an insertable support 400 that, when inserted into the receptacle 100, extends through the first chamber 216 of the main body 200. The support 400 includes an inlet 402 that permits passage of the specimen collector 204 into the main body 200 and an outlet 404 that restrictively permits passage of the specimen collector 204 into and out of the reservoir 210.

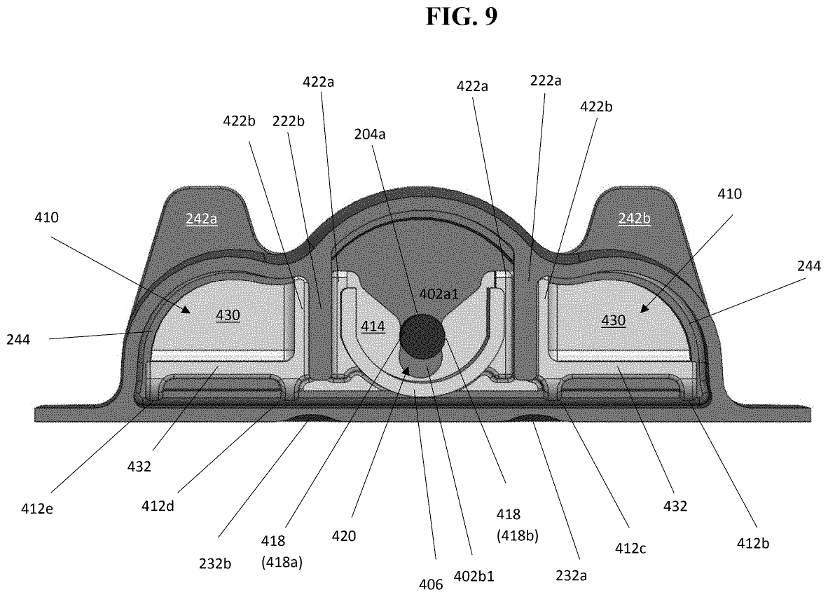

As shown in FIGS. 7A, 7B, and 9, the inlet 402 of the support 400 includes an entrance section 402a and a restriction section 4021). The restriction section 402b is disposed below the entrance section 402a in a direction intersecting the extending direction of the support 400. The outlet 404 includes a stopper section 404a and a wringer section 404b. The stopper section 404a is disposed in a direction facing the inlet 402 towards the proximal end side of the support 400 (and towards the main body 200 when inserted into the receptacle 100). The wringer section 404b is disposed on a distal end side of the support 400 (such that when the support 400 is inserted into the receptacle 100 the wringer section 404b faces the collection opening 262 of the reservoir 210 towards the distal end side of the reservoir 210).

The support 400 includes a guide channel 406 extending between the inlet 402 and the outlet 404 to guide the specimen collector 204 into and out of the receptacle 100 when the support 400 is inserted into the same.

As shown in FIGS. 4, 7A and 7B, the support 400 includes engagement members 408 configured to engage the interior wall portions (i.e., ribs 222a and 222b) disposed within the main body 200 to secure the support 400 within the main body 200 of receptacle 100.

The support 400 includes peripheral engagement sections 410 projecting outwardly from the engagement members 408 in a direction orthogonal to the extending direction of the support 400. The peripheral engagement sections 410 are configured to engage inner circumferential wall sections of the seal protrusion 244 when the cap 212 is attached to and seals the opening of the proximal end side of the main body 200. Thus, when the cap 212 engages the inner peripheral surface of the main body 200 to seal the opening of the same, the seal protrusion 244 is sandwiched between the peripheral engagement sections 410 and the inner peripheral surface of the main body 200. This connection prevents liquid and debris contained within the receptacle 100 from leaking outside of the receptacle 100.

As shown in FIGS. 5, 7A, 7B, 8, and 9, the support 400 includes a projection section 412 including a plurality of projections 412a, 412b, 412c, 412d, and 412e. The projections 412a, 412b, 412c, 412d. and 412e project outwardly from various areas of the support 400 for purposes of spacing and alignment of the support 400 within the receptacle 100. Projection 412a projects outwardly from an outer surface of the guide channel 406 adjacent to the outlet 404. The projection 412a is configured to contact the bottom surface 208b of the cover 208 to axially and concentrically align the outlet 404 with the collection opening 262 of the reservoir 210 when the support 400 is inserted into the receptacle 100. The axial alignment permits the specimen collector 204 to he readily and reliably inserted through the main body 200 and into the reservoir 210 to deposit the specimen therein for the targeted pathogen detection operation. In addition, the projection 412a provides a spacing gap 209 between the bottom surface 208b of the cover 208 and other portions of the support 400. This construction provides spacing between the cover 208 and the support 400 to permit the displacement of the cover 208 within the deformation zone 230 for crushing of the ampoules 202.

The projections 412b, 412c, 412d, and 412e project outwardly from outer side surfaces of the peripheral engagement sections 410 to space and align the support 400 within the receptacle 100. Projections 412b, 412c, 412d, and 412e project outwardly from an outer surface of a bottom wall 432 of each of the peripheral engagement sections 410 in a direction orthogonal to the extending direction of the guide track 406 and are spaced from the cover 208 when the support 400 is inserted into the receptacle 100. The projections 412b, 412c, 412d, and 412e are configured to contact inner peripheral surfaces of the seal protrusion 244 and facilitate alignment of the seal protrusion 244 with the opening of the main body 200 to readily and reliably insert the seal protrusion 244 into the opening of the main body 200 to seal the same.

The inlet 402 is a partition wall 414 of the support 400 extending upward from a proximal end side of the guide track 406. The partition wail 414 includes downward sloping inclined upper wall sections 414a and 414b. The downward sloping inclined upper wall sections 414a and 414b define a first opening 402a1 at an upper side of the partition wall 414 (at the entrance section 402a). A second opening 402b1 of the partition wall 414 is disposed below the first opening 402a in a direction intersecting the extending direction of the guide channel 406 (at the restriction section 402b).

The partition wall 414 further includes downward extending side wall sections 416a and 416b extending downward from lower side edges of the downward sloping inclined upper wall sections 414a and 414b. The second opening 402b1 is defined between the downward extending side wall sections 416a and 416b and is in fluid communication with the first opening 402a1. The downward extending side wall sections 416a and 416b also include a gate section 418 and a retention recess 420. The gate section 418 includes projections 418a and 418b projecting towards each other across the second opening 402b1 so as to narrow the second opening 402b1 at a position above the retention recess 420. The gate section 418 restricts inadvertent placement of the specimen collector 204 into the retention recess 420 during insertion of the same into the main body 200 for the targeted pathogen detection operation. On the other hand, the gate section 418 allows with appropriate downward force, the specimen collector 204 to pass through the gate section 418 i.e., to pass between the two projections 418a and 418b, to be retained in the retention recess 420 (and prevented from being easily pulled out).

Such a construction permits the user to not only retain the contaminated specimen collector 204 within the receptacle 100, but to also safely discard the contaminated specimen collector 204 with the used receptacle 100 after testing is complete. In particular, during the targeted pathogen detection operation and after the specimen is transferred to the reservoir 210, a user pulls the swab portion 204b from the reservoir 210, through the wringer section 404b, and into the opening 405 of the outlet 404 between the stopper section 404a and the wringer section 404b. Once secured in the opening 405, the user urges the handle portion 204a into the retention recess 420 to fixedly secure the same to the support 400. The specimen collector 204 is secured to the support 400 in this manner so as to be fixedly retained when the receptacle 100 is sealed with the cap 212 and to prevent the secured specimen collector 204 (and specifically the swab portion 204b) from re-entering the reservoir 210 once the receptacle 100 is sealed. Restricting the swab portion 204b from re-entering the reservoir 210 prevents the specimen collector 204 from serving as a “wick” that removes (i.e., absorbs) some amount of the reaction fluid from the reservoir 210 and promotes evaporation of the same during an incubation period. This design enables a sufficient amount of the reaction fluid to remain in the reservoir to ensure accuracy of the test.

While the swab portion 204b is described as being secured in the opening 405, it is contemplated that the swab portion 204b may also be considered to be secured when it abuts the stopper section 404a located at the entrance of the opening 405 on the proximal end side of the outlet 404.

The retention recess 420, which is provided at the proximal end side of the support 400 (and main body 200 when the support 400 is inserted into the receptacle 100), is spaced from the outlet 404 which is provided at the distal end side of the support 400 (and main body 200 when the support 400 is inserted into the receptacle 100), to permit reliable breakage of the handle portion 204a when the handle portion 204a is secured within the retention recess 420. With this arrangement, an upper end of the handle portion 204a is broken off and discarded from the remaining portions of the specimen collector 204 still secured to the support 400. The cap 212 may then be secured to the proximal end side of the main body 200 with the remaining portions of the specimen collector 204 retained therein for completion of the targeted pathogen detection operation and safe disposal of the contaminated specimen collector 204/used receptacle 100 after testing is complete.

As shown in FIGS. 4, 5, 7A, 7B, 8, and 9, the engagement members 408 each include a first side wall 422a and a second side wall 422b spaced from each other to define therebetween a slotted opening 424. The first side wall 422a. and the second side wall 422b are disposed in a parallel manner and project in the extending direction of the guide channel 406 for engaging within the slotted opening 424 the ribs 222a and 222b disposed within the receptacle 100. It is contemplated that the engagement may be friction fit and/or snap fit engagements, or any other releasable engaging means without departing from the spirit and scope of the application.

The peripheral engagement sections 410 each include a back wall 430 and the bottom wall 432 extending outward from each engagement member 408 in the direction orthogonal to the extending direction of the guide track 406. The back wall 430 is configured with an outer circumferential wall 431 contoured for engaging the inner circumferential wall sections of the seal protrusion 244. The back wall 430 extends outward from the second wall 422b in the direction orthogonal to the guide track 406. The back wall 430 also extends upward at the proximal end side of the guide track 406 in a manner similar to the partition wall 414. Each bottom wall 432 projects from a lower end of each back wall 430 (in the extending direction of the guide channel 400 such that when the support 400 is inserted into the main body 200 the bottom walls 432 project into the second and third Chambers 218 and 220, respectively. The bottom walls 432 project a distance equal to a projecting distance of each second wall 422b. The configuration of the back walls 430 and the bottom walls 432 restricts movement of the ampoules 202 toward the opening of the main body 200, as well as restricts shards of glass or plastic from a ruptured ampoule 202 (or other debris) from exiting the receptacle 100 and potentially harming a user if the cap 212 is inadvertently (or purposely) removed from a closed state on the main body 200. The configuration of the back walls 430 and the bottom walls 432 also restricts reaction fluid or transfer medium from leaking out of the main body 200 when the cap 212 is removed and can serve as a splash guard to a user opening the receptacle 100 for use and/or inspection of the interior of the same.

As shown in FIGS. 7A, 7B, and 8, the guide channel 406 projects from the partition wall 414 between each second wall 422b of the engagement members 408 and includes a track surface 406a flanked on opposite sides by and a first embankment wall 407a and a second embankment wall 407b. The guide channel 406 projects in the extending direction of the same beyond the projecting distance of the bottom wall 432 and the second wall 422b. In embodiments, the track surface 406a is arcuate although it is contemplated that the track surface may be flat, v-shaped, multi-grooved, etc. so long as the surface is capable of guiding the specimen collector 204 thereon towards the outlet 404 or supporting the specimen collector 204 from a bottom side thereof. It is contemplated that the support 400 may have a plurality of guide tracks and/or guide surfaces to permit insertion of multiple specimen collectors through multiple outlets 404 into a corresponding number of reservoirs 210 of the receptacle 100.

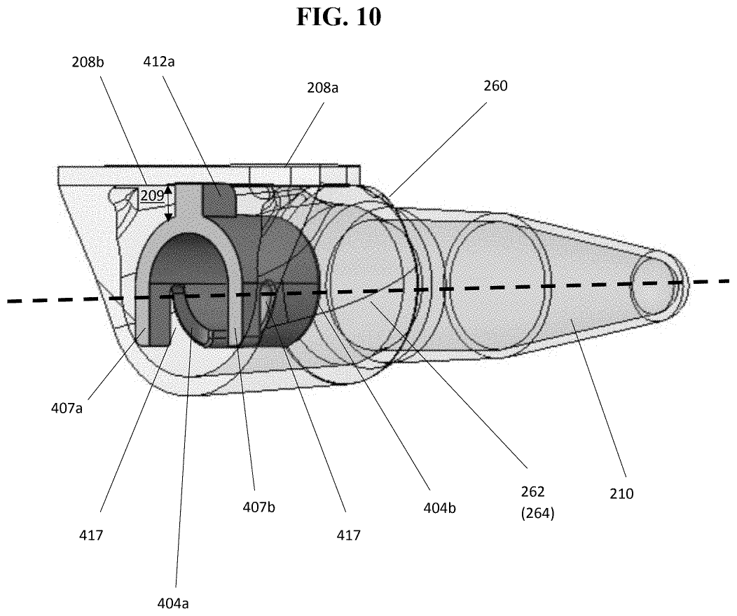

As also shown in FIGS. 7A, 7B, 8, and 10 the first embankment wall 407a and the second embankment wall 407b each include a cut part 417a and 417b and the outlet 404 is integrally formed at the cut parts 417a and 417b. As will be discussed in further detail below, the cut parts 417a and 417b may serve as relief openings in which undesirable debris shed from the swab portion 204b may pass through to an outer circumferential surface of one or both of the embankment walls 407a and 407b and the ribs 222a and 222b, and/or permit the debris to be wedged between the bottom surface 208b of the cover 208 and the outer surface of the guide channel 406.

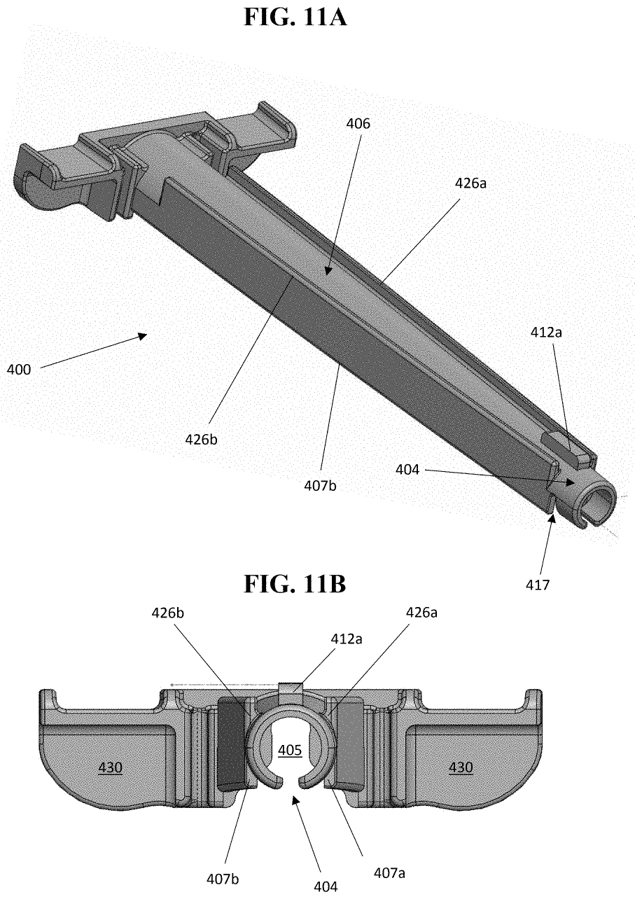

In embodiments and as shown in FIGS. 11A and 11B, the support 400 may also include restriction plates 426a and 426b provided on opposite sides of the guide channel 406 to restrict shards of glass or plastic from ruptured ampoules 202 from flowing with the released reagents into the reservoir 210. In particular, the restriction plates 426a and 426b project on opposite sides of the guide channel 406 from the first and second embankment walls 407a and 407, respectively, so as to project from the outer surface of guide channel 406 in the projecting direction of the projection section 412a. With such a construction, the restriction plates 426a and 426b project into the spacing gap 209 provided between the support 400 and the bottom surface 208b of the cover 208. Therefore, displacement of the cover 208 within the deformation zone 230 is still permitted.

Due to their height and extending length along the support 400, when the support 400 is inserted into the receptacle 100, the restriction plates 426a and 426b also function with the ribs 222a and 222b to retain the ampoules 202 in their respective chambers (during manufacture/assembly of the receptacle 100, during transport and storage, and during operational handling prior to rupturing). The restriction plates 426a and 426b further function to restrict shards of glass or plastic from ruptured ampoules 202 from entering into other portions of the receptacle 100.

In embodiments the restriction plates 426a and 426b extend in the extending direction of the support 400 from the proximal end side thereof to the cut parts 417a and 417b. When the support 400 is inserted into the main body 200 of the receptacle, the restriction plates 426a and 426b partially overlap outlet respective openings of the flow channels 218a and 220a that communicate the second and third chambers 218, 220 with the distal end side of the first chamber 216. The partial overlap of the restriction plates 426a and 426b with the flow channels 218a and 220a narrows the openings of the same so as to permit flow of the reagents while restricting the shards of glass or plastic from the ruptured ampoules 202 from also flowing into the reservoir 210. Thus, the amount of debris entering into the reservoir 210 can be minimized during the targeted pathogen detection operation and the accuracy of the operation enhanced.

As shown in FIGS. 7A, 7B, 8, and 10, the outlet 404 includes a first arcuate wall 404a1 and a second arcuate wall 404a2 that are resilient or flexible cut-out sections of the first embankment wall 407a and the second embankment wall 407b that curve inwardly from the cut parts 417a and 417b towards the guide track 406 relative to the first embankment wall 407a and the second embankment wall 407b. The first arcuate wall 404a1 and the second arcuate wall 404a2 are configured in an open ring shape having a through hole 405 to permit passage of the specimen collector 204. The first arcuate wall 404a1 and the second arcuate wall 404a2 curve toward each other in a semi-circular manner and define, between upper end surfaces 404a1′ and 404b1′ of the arcuate walls 404a1 and 404a2, a spacing gap 405a on a top side of the outlet 404. The spacing gap 405a is aligned with the projection 412a on opposite sides of the outlet 404 in a radial direction of the opening 405. With the spacing gap 405a, the first arcuate wall 404a1 and the second arcuate wall 404a2 are resiliently flexible between a flexed state and an un-flexed state. With such a construction, the outlet 404 is sized to accommodate a variety of specimen collector shapes and sizes including specimen collectors 204 having swab diameters larger than a diameter of the outlet 404 in the un-flexed state.

The first arcuate wall 404a1 and the second arcuate wall 404a2 define the stopper section 404a on a proximal end side of the outlet 404 and the wringer section 404b on a distal end side thereof. The stopper section 404a is defined by outer circumferential surfaces of the first arcuate wall 404a1 and the second arcuate wall 404a2 facing the proximal end side of the support 400 and define an entrance section of the opening 405. Because the outlet 404 is sized to be smaller than the swab diameter of an inserted specimen collector 204, the stopper section 404a is configured to shed undesirable debris on the swab portion 204b that may have accompanied the specimen during collection. When the swab portion 204b of the specimen collector 204 contacts the stopper section 404a and a force acting on the specimen collector 204 pushes the swab portion 204b through the opening 405 of the outlet 404, the swab portion 204b compresses so as to slide/pass through the stopper section 404a, through the opening 405 and into the reservoir 210. In doing so, the undesirable debris collected on the swab portion 204b is prevented from passing through the opening 405 and instead urged rearward in the guide channel 406 to disperse or collect, e.g., onto the track surface 406a. and/or wedge between an outer circumferential surface of one or both of the embankment walls 407a and 407b, the cut parts 417a and 417b, and the ribs 222a and 222b, and/or wedge between the bottom surface 208b of the cover 208 and the outer surface of the guide channel 406 due to the spacing gap 209 created by the projection 412a. Thus, the amount of debris entering into the reservoir 210 can be minimized during the targeted pathogen detection operation.

In addition, because the diameter of the outlet 404 is smaller than the swab diameter of the specimen collector 204, when no force is applied to the specimen collector 204 to push the same into the reservoir 210, the swab portion 204b of the specimen collector 204 may abut the stopper section 404a, i.e., contact the outer circumferential surfaces of the first arcuate wall 404a1 and the second arcuate wall 404a2 facing the proximal end side of the support 400, to prevent the specimen collector 204 from entering or re-entering the reservoir 210.

With this arrangement, the stopper section 404a restricts the specimen collector 204 from entering the reservoir 210 during and after the reaction. The stopper section 404a thus prevents the specimen collector 204 from absorbing reaction fluid during the targeted pathogen detection operation, which would otherwise result in the possibility of an insufficient sample amount being held in the reservoir 210 and increase the susceptibility of the reaction fluid to undesirable evaporation during an incubation cycle of the detection operation. In other words, the stopper section 404a serves to keep the specimen collector 204 out of the reservoir 210 during and after the reaction thereby preventing unnecessary depletion of the sample amount or reaction fluid in the reservoir 210 necessary for accurately completing the colorimetric reaction and analysis.

In a similar manner, the wringer section 404b is defined by outer circumferential surfaces of the first arcuate wall 404a1 and the second arcuate wall 404a2 provided on the opposite side of the outlet 404 from the stopper section 404a and facing the reservoir 210. When the swab portion 204b exits the reservoir 210 after immersion of the specimen into the transfer medium, a force acting on the specimen collector 204 pulls the swab portion 204b toward the opening 405 of the outlet 404 to contact the wringer section 404b where the swab portion 204b is then compressed so as to slide/pass back through the opening 405 and back onto the guide track 406. In doing so, specimen laden transfer medium absorbed into the swab portion 204b while in the reservoir 210 is wrung out or squeezed back into the reservoir 210 so that a sufficient volume of specimen laden transfer medium is prevented from passing back through the opening 405 and left in the reservoir 210 to react with the reagents. Thus, the amount of specimen laden transfer medium remaining in the reservoir 210 is maximized for completing the colorimetric reaction and analysis during the targeted pathogen detection operation.

It is noted that the arrangement of the various structural elements described above is not limited to the specific configuration depicted and may be modified in any suitable manner without departing from the spirit and the scope of the application.

An operation of detecting a targeted pathogen using the above-described receptacle 100 will now be described.

Initially and as shown in FIG. 1 the receptacle 100 is supplied with the reagent containing ampoules 202. In particular, the receptacle 100 is supplied with two reagent containing ampoules 202 housed in each of the second and third chambers 218 and 220 in the main body 200. The receptacle 100 is also supplied with the transfer medium, e.g., saline, a viral transport medium, etc. The reagent containing ampoules 202 and the transfer medium may be pre-supplied (or packaged) together in the receptacle 100 prior to use, although it is contemplated that the transfer medium may be pipetted into the receptacle 100 just prior to insertion of the specimen collector 704 containing the specimen.

As shown in 12A, when a specimen is collected by the specimen collector 204, the cap 212 is removed from the main body 200 in S101 to expose the proximal end side opening thereof to permit the specimen collector 204 containing the specimen to be inserted into the main body 200.

In S102, the specimen collector 204 containing the specimen is then received by or inserted into the main body 200 through the entrance section 402a of the inlet 402, along the track surface 406a of guide channel 406 towards the stopper section 404a.

In S103, the specimen collector 204 is pushed through the stopper section 404a of the outlet 404, through the opening 405, and out of the wringer section 404b into the reservoir 210. It is noted that during this operation the receptacle 100 may be at least slightly inclined downward such that the reservoir 210 is pointed downward and in a direction extending away from the user. Such orientation permits the transfer medium (if not already retained in the reservoir 210) to flow into the reservoir 210 prior to the insertion of the specimen collector 204 into the same and enable a user to readily insert the specimen collector 204 through the entrance section 402a into the opening of the main body 200 towards the reservoir 210.

In S104, when the specimen collector 204 containing the specimen is received into the reservoir 210, the swab portion 204b of the specimen collector 204 is immersed in the transfer medium within the reservoir 210 to transfer the specimen from the swab portion 204b into the transfer medium to create the specimen laden transfer medium.

In S105, once the transfer is complete, the specimen collector 204 is then pulled back through the wringer section 404b, through the opening 405, and out of the stopper section 404a to the guide channel 406 to abut against the stopper section 404b. In embodiments, however, it is contemplated that the swab portion 204b of the specimen collector 204 may instead be at least partially retained in the opening 405 due to the interference fit.

In either state and as shown in FIGS. 2 and 8, a proximal side of the handle portion 204a of the specimen collector 204 is then inserted into or received by the retention recess 420 to be locked therein preventing both lateral and vertical adjustment of the handle portion 204a. In particular, when the specimen collector 204 passes through or is retained within the stopper section 404a after transferring the specimen into the transfer medium in the reservoir 210, a breakable portion 204a1 i.e., a portion of the handle 204a extending from an intersecting point P at the partition wall 414 towards the outside of the receptacle 100 is forced downward from the entrance section 402a towards the gate section 418, while a retained portion 204a2 of the handle 204a extending from the intersecting point P at the partition wall 414 towards the distal side end of the main body 200 remains supported within the support 400.

With sufficient force, the breakable portion 204a1 of the handle portion 204a is urged through the resilient protrusions 418a and 418b to be locked in the retention recess 420 in an interference fit manner. Once locked, a force acts on breakable portion 204a1 to create a bending moment at the contact point between the breakable portion 204a1 and a side wall surface of the retention recess 420. The force causes the breakable portion 204a1 to snap off due to bending stress failure.

In S106, with the breakable portion 204a1 removed, the cap 212 is then free to be reconnected to the main body 200 to seal the used specimen collector 204 within the receptacle 100 between the cap 212 and the outlet 404 of the support 400. Sealing the receptacle 100 in this manner minimizes contamination to users and secures any biohazard within the receptacle 100 during and after testing. With the biohazard securely contained within the receptacle 100, the used receptacle 100 may be readily disposed of in a safe and sanitary manner without compromising the health and safety of users and others that may be exposed to the receptacle 100 after use.

In S107, with the receptacle 100 sealed and in the at least slightly downward inclined orientation, a force is applied to or received by the deformation zone 230 of the cover 208 to deform the cover 208. The cover 208 deforms inwardly and downwardly within the central region of the main body 200 such that the bottom surface 208b of the cover 208 (disposed within the interior of the main body 200) contacts the frangible reagent containing ampoules 202. Contact of the bottom surface 208b of the cover 208 with the ampoules 202 pins the ampoules 202 between the bottom inner surfaces 224 of the second and third chambers 218 and 220 and the bottom surface 208b of the cover 208. With sufficient force acting on the cover 208, the clamped ampoules 202 are ruptured and release the reagents to flow through the flow channels 218a, and 220a into the reservoir 210 with the specimen laden transfer medium.

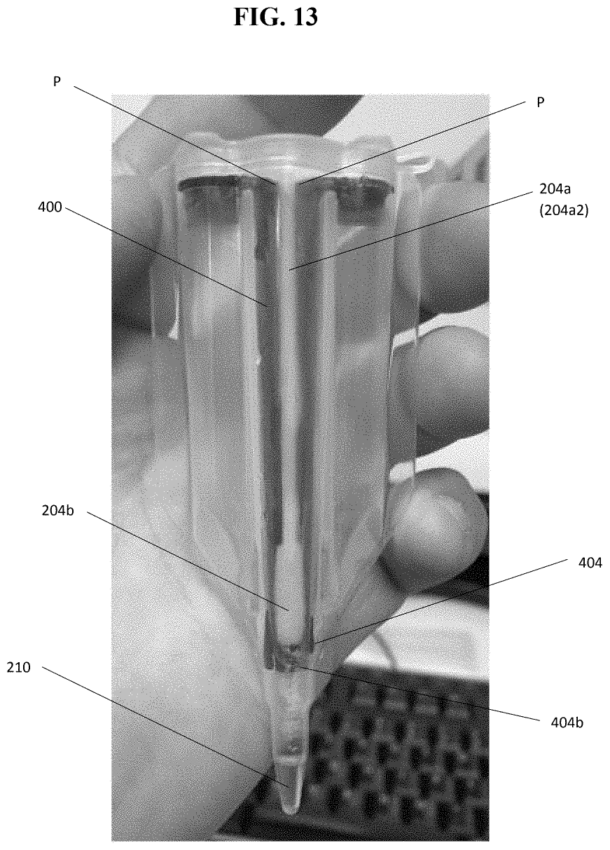

In S108 and as shown in FIG. 13, the reagents are mixed with the specimen laden transfer medium and the mixture is agitated (via, e.g., a shaking of the receptacle 100) to incite a colorimetric reaction in the reservoir 210.

To complete the colorimetric analysis, in S109A a visual inspection of the reaction fluid color, along with a comparison against a color chart, may confirm the presence or absence of the targeted pathogen.

In other embodiments, to complete the colorimetric analysis, the colorimetric reaction within the reservoir 210 is prepared to undergo an incubation cycle, e.g., a heat cycle or a temperature stabilization cycle in which the reaction fluid is maintained at a constant temperature (e.g., ambient temperature or other temperature above or below ambient temperature) for a predetermined amount of time to stabilize the reaction fluid for results interpretation.

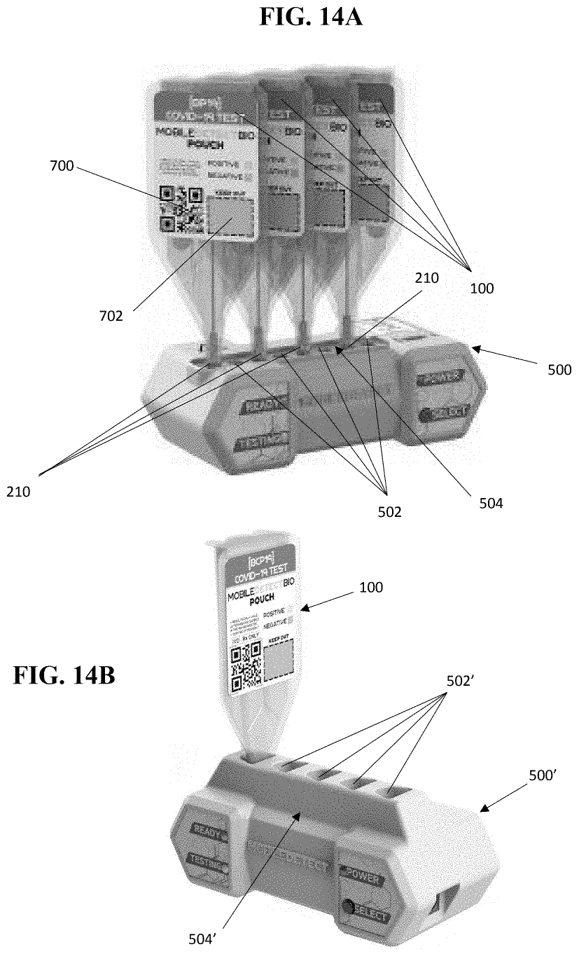

In S109B and as shown in FIGS. 14A and 14B, when reaction fluid in the reservoir 210 undergoes a heat cycle, one or more receptacles 100 containing a colorimetric reaction are placed into a pre-heated incubator 500 (500′). In particular, the reservoir 210 containing the colorimetric reaction is inserted into a hole 502 (502′) provided at an upper surface of a heat block 504 (504′) on the incubator 500 (500′). The holes 502′ shown in FIG. 14B are configured such that the receptacles 100 can only be inserted into the incubator 500′ one way. Such a construction of the holes 502′ prevents insertion errors and facilitates alignment of multiple inserted receptacles 100 for simultaneous incubation.

The colorimetric reaction is heated for a predetermined amount of time (i.e., for an incubation period). When the incubation period is complete, the one or more receptacles 100 is removed from the incubator 500 (500′). Once the chemistry of the reaction reaches a thermal equilibrium point (e.g., the reaction clearly indicates a color for either positive or negative results), in S110 the colorimetric reaction is ready for result interpretation.

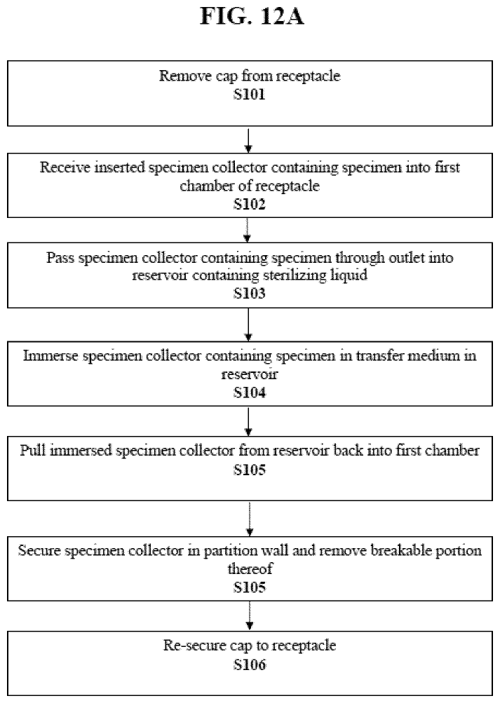

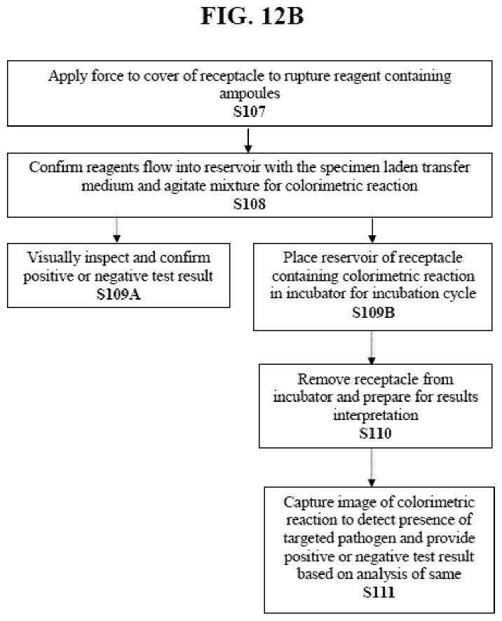

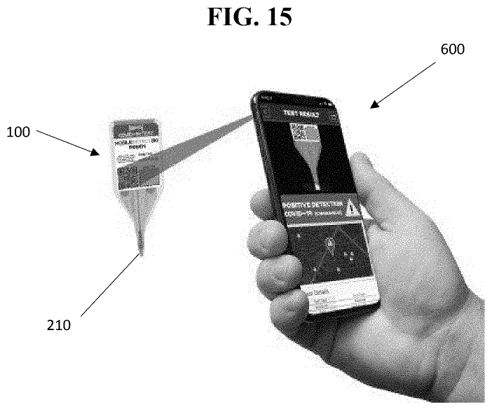

At that time, the application on a computing device (e.g., smartphone 600) will interpret the color of the reaction fluid shown in the reservoir 210. As shown in FIGS. 12B and 15, and described in S111, the smartphone 600 application will prepare to capture an image that registers a location of indicia 700 (e.g., a OR code) while locating the colorimetric reaction in the reservoir 210 and identifying a color calibration area 702. The calibration area 702 serves to baseline a color spectrum, which can vary due to environmental and lighting conditions (e.g., insufficient and/or oversaturation of natural lighting, simultaneous flashing of superficial lighting having varying colors and brightness levels, shadows, etc. . . ) to prevent distortion of the true color of the reaction fluid captured in the image thereby enhancing the accuracy of the results of the colorimetric analysis performed by the application. Thus, the smartphone 600 application automatically analyzes the color of the colorimetric reaction in the reservoir 210 to detect the presence of the targeted pathogen and to provide a presumptive positive or negative test result for the same on a display of the smartphone 600. The results can then be transmitted via email, text, etc. and may include images of the patient, test location, notes, voice memos and other collection information pertinent for monitoring and further analysis by the appropriate regulatory body.

Although the apparatus and method for the detection of targeted pathogens has been described with reference to several exemplary embodiments, it is understood that the words that have been used are words of description and illustration, rather than words of limitation. Changes may be made within the purview of the appended claims, as presently stated and as amended, without departing from the scope and spirit of the products and methods for the detection of targeted pathogens in its aspects. Although the products and methods for the detection of targeted pathogens has been described with reference to particular means, materials and embodiments, the products and methods for the detection of targeted pathogens are not intended to be limited to the particulars disclosed; rather the products and methods for the detection of targeted pathogens extend to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent, to those of skill in the art upon reviewing the disclosure. Other embodiments may be utilized and derived from the disclosure, such that structural and logical substitutions and changes may be made without departing from the scope and spirit of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will he apparent to those of skill in the art upon reviewing the description.

The Abstract of the Disclosure complies with the relevant patent rules and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to make or use the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing Detailed Description.

What is claimed is:

1. A receptacle for detecting a targeted pathogen, comprising:
a main body configured to accommodate reagent containing ampoules and a specimen collector;
a reservoir provided at a distal end side of the main body configured to receive reagents from ruptured reagent containing ampoules; and
a support extending through the main body in an extending direction of the receptacle, the support including an inlet that permits passage of the specimen collector into the main body and an outlet that restrictively permits passage of the specimen collector into and out of the reservoir.

2. The receptacle of claim 1, wherein
the main body defines a first chamber, a second chamber, and a third chamber,
the second chamber and the third chamber are disposed on opposite sides of the first chamber and are each configured to accommodate one of the reagent containing ampoules,
the support is disposed in the first chamber, and
the first chamber, the second chamber and the third chamber are in fluid communication with the reservoir such that when the reagent containing ampoules accommodated in each of the second chamber and the third chamber are ruptured, the reagents are respectively released to flow into the reservoir.

3. The receptacle of claim 1, wherein
the inlet of the support includes an entrance section and a restriction section, the restriction section being disposed below the entrance section in a direction intersecting the extending direction of the receptacle, and
the outlet includes a stopper section and a wringer section, the stopper section being disposed in a direction facing a proximal end side of the main body and the wringer section being disposed in a direction facing a proximal end side of the reservoir.

4. The receptacle of claim 1, wherein
the support includes a guide channel extending between the inlet and the outlet that is configured to guide the specimen collector through the main body and into and Gut of the reservoir.

5. The receptacle of claim 1, wherein
the support is releasably attached to the main body so as to be insertable into and removable out of the receptacle.

6. The receptacle of claim 1, wherein
the support includes:
engagement members configured to engage a wall section disposed within the main body to secure the support within the main body;
peripheral engagement sections projecting outwardly from each of the engagement members and configured to engage inner circumferential wall sections of a cap to secure the cap to the main body; and
a projection projecting outwardly from the outlet and configured to contact the main body to align the outlet with an opening of the reservoir.

7. The receptacle of claim 1, wherein
the main body includes a cover that is disposed along an upper side of the main body,
the cover includes a deformation zone that permits resilient deformation of the cover toward an interior of the main body,
the deformation zone includes materially thinned portions of the cover, and
when the reagent containing ampoules are accommodated in the main body and an input force acting on the cover ruptures the reagent containing ampoules, the deformation zone permits displacement of the cover towards the main body to trigger release of the reagents from the main body into the reservoir.

8. The receptacle of claim 1, further comprising:
a removable cap provided to seal a proximal end side opening of the main body, wherein
the cap includes a stand section configured to stabilize placement of the receptacle in a vertically extending orientation and in a horizontally extending orientation.

9. The receptacle of claim 14, wherein
the support includes restriction plates provided on opposite sides of the guide channel to restrict the ruptured reagent containing ampoules from flowing with the released reagents from the main body into the reservoir.

10. A method of detecting a targeted pathogen, comprising:
supplying the receptacle of claim 1 with a transfer medium and the reagent containing ampoules accommodated in the main body;
inserting a specimen col lector containing a specimen into the main body through the inlet and the outlet of the support and into the reservoir to immerse the specimen in the transfer medium;
removing the specimen collector from the reservoir through the outlet;
applying an input force to deform the main body to rupture the reagent containing ampoules and release the reagents into the reservoir with the specimen immersed in the transfer medium;
agitating the mixture of the released reagents, the specimen, and the transfer medium to incite a colorimetric reaction in the reservoir; and
detecting the targeted pathogen based on a color of the colorimetric reaction.

11. The method of detecting the targeted pathogen of claim 10, wherein
the inlet of the support includes an entrance section and a restriction section, the restriction section being disposed below the entrance section in a direction intersecting the extending direction of the receptacle,
the outlet includes a stopper section and a wringer section, the stopper section being disposed in a direction facing a proximal end side of the main body and the wringer section being disposed in a direction facing a proximal end side of the reservoir,
during the inserting of the specimen collector into the main body, the specimen collector passes through the entrance section of the inlet and the stopper section of the outlet, and
during the removing of the specimen collector from the reservoir, the specimen collector is pulled through the wringer section of the outlet so as to wring excess specimen laden transfer medium from the specimen collector back into the reservoir.

12. The method of detecting the targeted pathogen of claim 10, Wherein
the main body includes a cover that is disposed along an upper side of the main body, the cover includes a deformation zone that permits resilient deformation of the cover toward an interior of the main body,
the deformation zone includes materially thinned portions of the cover, and
during the applying of the input force to deform the main body, the input force acts on the cover such that the deformation zone permits displacement of the cover towards the main body to trigger the release of the reagents from the main body into the reservoir.

13. The method of detecting the targeted pathogen of claim 10, wherein
the receptacle further comprises a removable cap provided to seal a proximal end side opening of the main body,
prior to the inserting of the specimen collector containing the specimen into the receptacle, the method further comprising:
removing the cap from the main body to expose the proximal end side opening thereof to permit the inserting of the specimen collector containing the specimen into the main body; and
after the removing of the specimen collector from the reservoir through the outlet, the method further comprising:
retaining the specimen collector between the inlet and the outlet of the support; and
securing the cap to the main body to seal the proximal end side opening and the specimen collector within the receptacle.

14. The method of detecting the targeted pathogen of claim 11, wherein
after specimen collector is pulled through the wringer section, locking the specimen collector into the restriction section of the inlet of the support, removing a breakable portion of the specimen collector extending from the restriction portion in the extending direction of the receptacle, and securing a cap to the main body to seal the receptacle and retain the specimen collector between the cap and the outlet of the support.

15. The method of detecting the targeted pathogen of claim I 0, wherein
after the removing of the specimen collector from the reservoir through the outlet, the method further comprising:
securing the specimen collector to the support;
sealing the receptacle with the specimen collector retained therein;
inserting the reservoir into an incubator and heating the colorimetric reaction for a pre-determined amount of time;
removing the reservoir from the incubator;
capturing, with an image processor of a computing device, an image of the colorimetric reaction in the reservoir to facilitate an automated colorimetric analysis performed by the computing device for detecting the targeted pathogen; and
displaying, on a display of the computing device, a result of the colorimetric analysis by the computing device indicating a presence of or an absence of the targeted pathogen.

16. A support removably insertable into a receptacle for detecting a targeted pathogen, the support comprising:
an inlet that permits passage of a specimen collector into the receptacle and an outlet that restrictively permits passage of the specimen collector further into the receptacle;
engagement members disposed adjacent to the inlet and configured to engage wall sections disposed within the receptacle;
peripheral engagement sections projecting outwardly from each of the engagement members and configured to engage inner circumferential wall sections of a cap of the receptacle; and
a guide channel extending between and connecting the inlet to the outlet to permit passage of the specimen collector into and out of the receptacle.

17. The support of claim 16. wherein
the inlet is a partition wall of the support and includes an entrance section and a restriction section,
the entrance section is a first opening of the partition wall and is disposed at an upper side of the partition wall,
the restriction section is a second opening of the partition wall and is disposed below the entrance section in a direction intersecting the extending direction of the guide channel,
the first opening is defined by downward sloping inclined upper wall sections of the partition wall, and
the second opening is in fluid communication with the first opening, is defined by downward extending side wall sections of the partition wall, and includes a gate section and a retention recess.

18. The support of claim 16, wherein
the engagement members each include a first side wall and a second side wall spaced from each other to define therebetween a slotted opening,
the first side wall and the second side wall are disposed in a parallel manner and project in the extending direction of the guide channel for engaging within the slotted opening the wall sections disposed within the receptacle,
the peripheral engagement sections each include a back wall and a bottom wall adjacent to the second side wall of each of the engagement members,
the back wall is configured with an outer circumferential wall contoured for engaging inner circumferential wall sections of the cap of the receptacle,
the bottom wall includes an upper surface and projects in the extending direction of the guide channel, and
the guide channel projects from the inlet between the first side wall of each of the engagement members and includes a track surface flanked on opposite sides by a first embankment wall and a second embankment wall.

19. The support of claim 16, wherein
the outlet includes a first arcuate wail and a second arcuate wall configured in an open ring shape having a through hole to restrictively permit passage of the specimen collector therethrough, and
the first arcuate wall and the second arcuate wall define a stopper section on a proximal end side of the outlet and a wringer section on a distal end side thereof.

20. The support of claim 18, wherein
the first embankment wall and the second embankment wall each include a cut part and the outlet is integrally formed at the cut part,
the outlet includes a first arcuate wall and a second arcuate wall configured in an open ring shape axially aligned with the guide track to restrictively permit passage of the specimen collector therethrough, and
the first curved wall and the second curved wall define a stopper section on a proximal end side of the outlet and a wringer section on a distal end side thereof.
